(19) United States
(12) Patent Application Publication
(10) Pub. No.: US 2006/0063964 A1
(43) Pub. Date: Mar. 23, 2006

(54) Micro electromechanical machine-based ventricular assist apparatus

(76) Inventor: Richard J. Massen, Albuquerque, NM (US)

Correspondence Address: Luis M. Ortiz;Ortiz & Lopez, PLLC, Registered Patent Attorneys, P.O. Box 4484, Albuquerque, NM, 87196-4484, US

(21) Appl. No.: 10/923,357

(22) Filed: Aug. 20, 2004

(51) Int. Cl. A61N 1/362; A61M 1/10

(52) U.S. Cl. 600/16; 623/3.11

(57) ABSTRACT

A ventricular assist apparatus is disclosed, which is composed of a sheet of MEMS-based material wrapped around a failing heart, wherein the sheet of MEM-based material contracts or expands in order to support the failing heart and ventricular activities thereof. Additionally, such an apparatus can include a controller in communication with the sheet of MEMS-based material for controlling a contraction or an expansion of the sheet when the sheet is wrapped around the failing heart and a microprocessor in communication with the controller for processing data communicated to and from the controller. A pacemaker in communication with the sheet can be configured to include the controller and the microprocessor.

### TECHNICAL FIELD

Embodiments are generally related to ventricular assist devices. Embodiments are also related to devices for assisting the natural heart in operation thereof. Embodiments are additionally related to micro electromechanical systems (MEMS).

### BACKGROUND OF THE INVENTION

The natural human heart and accompanying circulatory system are critical components of the human body and systematically provide the needed nutrients and oxygen for the body. As such, the proper operation of the circulatory system, and particularly, the proper operation of the heart, is critical in the overall health and well being of a person. A physical ailment or condition which compromises the normal and healthy operation of the heart can therefore be particularly critical and may result in a condition which must be medically remedied.

Specifically, the natural heart, or rather the cardiac tissue of the heart, can fail for various reasons to a point where the heart can no longer provide sufficient circulation of blood for the body so that life can be maintained. To address the problem of a failing natural heart, solutions are offered to provide ways in which circulation of blood might be maintained.

Some solutions involve replacing the heart. Other solutions are directed to maintaining operation of the existing heart. One such solution has been to replace the existing natural heart in a patient with an artificial heart or a ventricular assist device. In using artificial hearts and/or assist devices, a particular problem stems from the fact that the materials used for the interior lining of the chambers of an artificial heart are in direct contact with the circulating blood. Such contact may enhance undesirable clotting of the blood, may cause a build-up of calcium, or may otherwise inhibit the blood's normal function. As a result, thromboembolism and hemolysis may occur. Additionally, the lining of an artificial heart or a ventricular assist device can crack, which inhibits performance, even when the crack is at a microscopic level. Moreover, these devices must be powered by a power source, which may be cumbersome and/or external to the body. Such drawbacks have limited use of artificial heart devices to applications having too brief of a time period to provide a real lasting benefit to the patient.

An alternative procedure also involves replacement of the heart and includes a transplant of a heart from another human or animal into the patient. The transplant procedure requires removing an existing organ (i.e. the natural heart) from the patient for substitution with another organ (i.e. another natural heart) from another human, or potentially, from an animal. Before replacing an existing organ with another, the substitute organ must be “matched” to the recipient, which can be, at best, difficult, time consuming, and expensive to accomplish. Furthermore, even if the transplanted organ matches the recipient, a risk exists that the recipient's body will still reject the transplanted organ and attack it as a foreign object. Moreover, the number of potential donor hearts is far less than the number of patients in need of a natural heart transplant. Although use of animal hearts would lessen the problem of having fewer donors than recipients, there is an enhanced concern with respect to the rejection of the animal heart.

In an effort to continue use of the existing natural heart of a patient, other attempts have been made to wrap skeletal muscle tissue around the natural heart to use as an auxiliary contraction mechanism so that the heart may pump. As currently used, skeletal muscle cannot alone typically provide sufficient and sustained pumping power for maintaining circulation of blood through the circulatory system of the body. This is especially true for those patients with severe heart failure.

Another system developed for use with an existing heart for sustaining the circulatory function and pumping action of the heart, is an external bypass system, such as a cardiopulmonary (heart-lung) machine. Typically, bypass systems of this type are complex and large, and, as such, are limited to short term use, such as in an operating room during surgery, or when maintaining the circulation of a patient while awaiting receipt of a transplant heart. The size and complexity effectively prohibit use of bypass systems as a long-term solution, as they are rarely portable devices. Furthermore, long-term use of a heart-lung machine can damage the blood cells and blood borne products, resulting in post surgical complications such as bleeding, thromboembolism function, and increased risk of infection.

Still another solution for maintaining the existing natural heart as the pumping device involves enveloping a substantial portion of the natural heart, such as the entire left and right ventricles, with a pumping device for rhythmic compression. That is, the exterior wall surfaces of the heart are contacted and the heart walls are compressed to change the volume of the heart and thereby pump blood out of the chambers. Although somewhat effective as a short-term treatment, the pumping device has not been suitable for long-term use.

Typically, with such compression devices, a vacuum pressure is needed to overcome cardiac tissue/wall stiffness, so that the heart chambers can return to their original volume and refill with blood. This “active filling” of the chambers with blood limits the ability of the pumping device to respond to the need for adjustments in the blood volume pumped through the natural heart, and can adversely affect the circulation of blood to the coronary arteries. Furthermore, natural heart valves between the chambers of the heart and leaching into and out of the heart are quite sensitive to wall and annular distortion. The movement patterns that reduce a chamber's volume and distort the heart walls may not necessarily facilitate valve closure (which can lead to valve leakage).

Therefore, mechanical pumping of the heart, such as through mechanical compression of the ventricles, must address these issues and concerns in order to establish the efficacy of long term mechanical or mechanically assisted pumping. Specifically, the ventricles must rapidly and passively refill at low physiologic pressures, and the valve functions must be physiologically adequate. The mechanical device also must not impair the myocardial blood flow of the heart. Still further, the left and right ventricle pressure independence must be maintained within the heart.

Another major obstacle with long term use of such pumping devices is the deleterious effect of forceful contact of different parts of the living internal heart surface (endocardium), one against another, due to lack of precise control of wall actuation. In certain cases, this cooptation of endocardium tissue is probably necessary for a device that encompasses both ventricles to produce independent output pressures from the left and right ventricles. However, it can compromise the integrity of the living endothelium.

Mechanical ventricular wall actuation has shown promise, despite the issues noted above. As such, devices have been invented for mechanically assisting the pumping function of the heart, and specifically for externally actuating a heart wall, such as a ventricular wall, to assist in such pumping functions.

One particular type of mechanical ventricular actuation device that has been developed is a Left Ventricular Assist Device (LVAD), which is designed to support the failing heart. Such a device must augment systolic function. Diastolic function must also be augments or at the very least, not worsened, while allowing blood flow between the right and left ventricular portions of the heart. If the LVAD relies on a pump mechanism, the heart must still be able to beat 45 to 40 million times per year. The LVAD must therefore be durable and should function flawlessly or permit some degree of cardiac function in case of device failure. Such devices and/or systems must also permit a minimal risk for blood clot production and should be resistant to infection.

It is believed that a solution to the aforementioned problems associated with conventional ventricular assist devices involves the use of so-called micro electromechanical system (MEMS) technology. Micro Electro Mechanical System (MEMS) is a technology that implements mechanical and electrical parts, using semiconductor-processing techniques. A conventional MEMS device generally includes floating driving parts that are movable over a substrate in order for the device fabricated using MEMS technology to perform mechanical operations. In general, MEMS technology involves the integration of mechanical components, sensors, actuators and other electronic and/or mechanical elements on a common substrate (e.g., silicon) through the use of micro fabrication manufacturing techniques

### BRIEF SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

It is, therefore, one aspect of the present invention to provide for an improved ventricular assist device.

It is another aspect of the present invention to provide for devices and systems for assisting the natural heart in operation thereof.

It is yet a further aspect of the present invention to provide for a ventricular assist device and system that is composed sheet of MEMS-based material that can be wrapped around a failing heart to support ventricular activities thereof.

The aforementioned aspects of the invention and other objectives and advantages can now be achieved as described herein. A ventricular assist apparatus is disclosed, which is composed of a sheet of MEMS-based material wrapped around a failing heart, wherein the sheet of MEM-based material contracts or expands in order to support the failing heart and ventricular activities thereof. Additionally, such an apparatus can include a controller in communication with the sheet of MEMS-based material for controlling a contraction or an expansion of the sheet when the sheet is wrapped around the failing heart and a microprocessor in communication with the controller for processing data communicated to and from the controller. A pacemaker in communication with the sheet can be configured to include the controller and the microprocessor.

In general, the sheet comprises a plurality of MEMS elements linked to one another. Each MEMS element can be configured to include an embedded electrical polarity, which contributes to the generation of a force for the contraction or expansion of the sheet in order to support ventricular activities of a heart and prevent failure thereof when the sheet is wrapped around the heart.

The MEMS elements can be configured to include electrical conducting elements for generating electrical potentials at predefined times, thereby resulting in the aforementioned embedded polarity, which can contribute to the generation of a force for the contraction or expansion of the sheet to support ventricular activities of the heart and prevent failure thereof. The sheet therefore possesses a diastolic function and a systolic function in support of the ventricular activities of the heart. The diastolic function can be augmented by active relaxation of the sheet, while the systolic function can be augmented by myocarderial contraction of the sheet.

Additionally, each MEMS element among the plurality of MEMS elements of the sheet can contract toward one another in systole and away from one another by a reversal of poles thereof in diastole. Each MEMS element among the plurality of MEMS elements also can sequentially contract the heart horizontally and thereafter, vertically. Each MEMS element is therefore linkable, contractile, durable and electrically insulated to performance characteristics by design. The sheet can be configured from a flexible and/or a pliable material, and may be arranged as a sheath and/or in a mesh arrangement of the MEMS elements.

### BRIEF DESCRIPTION OF THE DRAWINGS

The accompanying figures, in which like reference numerals refer to identical or functionally-similar elements throughout the separate views and which are incorporated in and form a part of the specification, further illustrate the present invention and, together with the detailed description of the invention, serve to explain the principles of the present invention.

FIG. 1 illustrates a pictorial perspective view of a human heart whose ventricular activities can be supported and enhanced utilizing an embodiment of the present invention;

FIG. 2 illustrates a pictorial diagram of a human heart prior to and following a single-sheet of MEM-based material wrapped about the heart, in accordance with a preferred embodiment of the present invention;

FIG. 3 illustrates a pictorial diagram of a human heart wrapped with a sheath formed from a single-sheet of MEM-based material, in accordance with an alternative embodiment of the present invention;

### DETAILED DESCRIPTION OF THE INVENTION

The particular values and configurations discussed in these non-limiting examples can be varied and are cited merely to illustrate at least one embodiment of the present invention and are not intended to limit the scope of the invention.

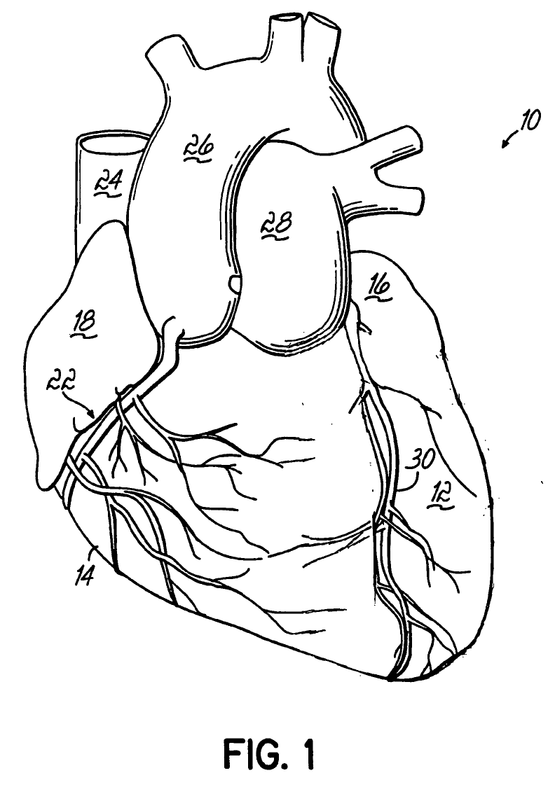

FIG. 1 illustrates a natural human heart 10, which is depicted in perspective, including a lower portion comprising two chambers, namely a left ventricle 12 and a right ventricle 14, which function primarily to supply the main pumping forces that propel blood through the circulatory system, including the pulmonary system (lungs) and the rest of the body, respectively. Note that heart 10 depicted in FIG. 1 is presented for generally illustrative and edification purposes only. Heart 10 also includes an upper portion having two chambers, a left atrium 16 and a right atrium 18, which primarily serve as entryways to the ventricles 12 or 14, and also assist in moving blood into the ventricles 12 or 14. The interventricular wall or septum of cardiac tissue separating the left and right ventricles 12 and 14, is defined externally by an interventricular groove 20 on the exterior wall of the natural heart 10. The atrioventricularvwall of cardiac tissue separating the lower ventricular region from the upper atrial region is defined by atrioventricular groove 22 on the exterior wall of the natural heart 10. The configuration and function of the heart is known to those skilled in this art.

Generally, the ventricles are in fluid communication with their respective atria through an atrioventricular valve in the interior volume defined by heart 10. More specifically, the left ventricle 12 is in fluid communication with the left atrium 16 through the mitral valve, while the right ventricle 14 is in fluid communication with the right atrium 18 through the tricuspid valve. Generally, the ventricles are in fluid communication with the circulatory system (i.e., the pulmonary and peripheral circulatory system) through semilunar valves. More specifically, the left ventricle 12 is in fluid communication with the aorta 26 of the peripheral circulatory system, through the aortic valve, while the right ventricle 14 is in fluid communication with the pulmonary artery 28 of the pulmonary, circulatory system through the pulmonic or pulmonary valve.

The heart basically acts like a pump. The left and right ventricles are separate, but share a common wall, or septum. The left ventricle has thicker walls and pumps blood into the systemic circulation of the body. The pumping action of the left ventricle is more forceful than that of the right ventricle, and the associated pressure achieved within the left ventricle is also greater than in the right ventricle. The right ventricle pumps blood into the pulmonary circulation, including the lungs. During operation, the left ventricle fills with blood in the portion of the cardiac cycle referred to as diastole. The left ventricle then ejects any blood in the part of the cardiac cycle referred to as systole. The volume of the left ventricle is largest during diastole, and smallest during systole. The heart chambers, particularly the ventricles, change in volume during pumping.

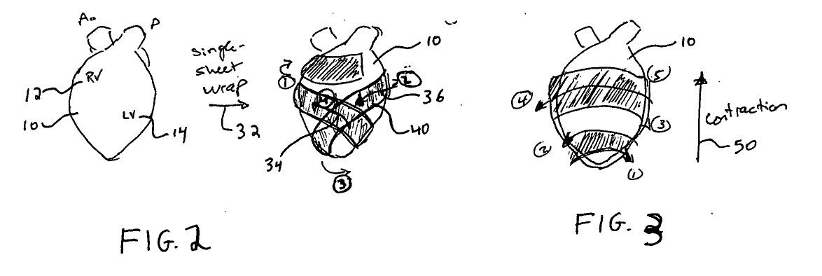

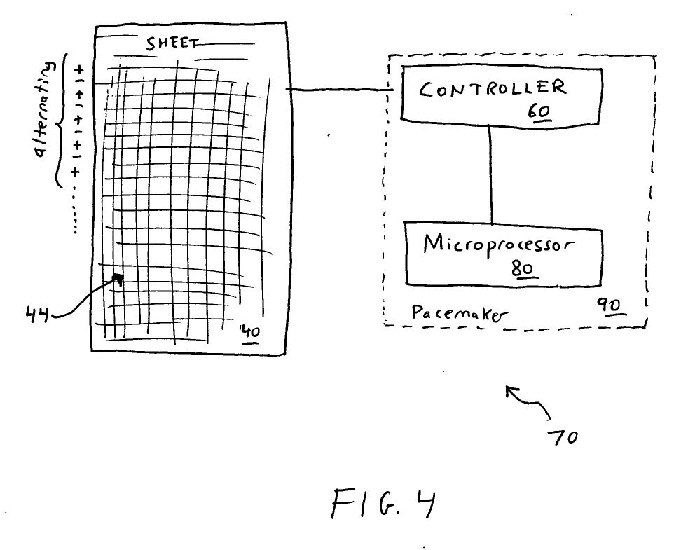

FIG. 2 illustrates a pictorial diagram of a human heart prior to and following a single-sheet 40 of MEM-based material wrapped about the heart, in accordance with a preferred embodiment of the present invention. Note that in FIGS. 2-3, identical or similar parts or elements are generally indicated by identical reference numerals. Thus, heart 10 depicted in FIG. 1 is also depicted in FIGS. 2-3. Left and right ventricles 12 and 14 are also shown in FIG. 2. Arrow 32 indicates a subsequent wrapping of heart 10 by sheet 40. Also indicated in FIG. 2 are five general requirements, including, as indicated at point 1, that the MEM-based material of sheet 40 is preferably composed of a group of (MEMS) elements linked to one another. As indicated at point 2, each MEMS element among the group of MEMS elements forming sheet 40 should possess an embedded electrical polarity, which contributes to the generation of a force for contraction or expansion by sheet 40 in order to support ventricular activities of heart 10 and prevent failure thereof when sheet 40 is wrapped around heart 10. As indicated at point 3, sheet 40 thus provides a contractile function. Relaxation occurs by reversing the electrical polarity in diastole. Arrows 24 and 36 illustrate such a contractile action. As indicated at point 4, each MEMS element among said plurality of MEMS elements composing sheet 40 is electrical insulated.

FIG. 3 illustrates a pictorial diagram of human hear 10 wrapped with a sheath formed from the single-sheet 40 of MEM-based material, in accordance with an alternative embodiment of the present invention. Note that points 1-5 indicated above with respect to FIG. 2 apply equally to the alternative embodiment depicted in FIG. 5. An additional point 5 should be noted in that the MEM-based elements of sheet 40 can be implemented at a nano-scale instead of a micro-scale. Sheet 40 depicted in FIG. 3 is therefore implemented as a sheath rather than a single-sheet as indicated in FIGS. 1-2. Note that points 1-4 depicted in FIG. 2 and points 1-5 indicated in FIG. 3 can also indicate a contractile sequence for support of heart 10. Arrow 50 in FIG. 2 represents contraction provided by sheet 40.

FIG. 4 illustrates a ventricular assist apparatus 70, which can be implemented in accordance with an alternative embodiment of the present invention. Note that in FIGS. 1-4, identical or similar parts are generally indicated by identical reference numerals. Sheet 40 therefore can be configured as a sheet composed of a plurality of MEMS elements 44 linked to one another. Each MEMS element among the group of MEMS elements 44 includes an embedded polarity, which contributes to the generation of a force for contraction or expansion by sheet 40 in order to support ventricular activities of heart 10 and prevent failure thereof when sheet 40 is wrapped around heart 10.

The MEMS elements 44 comprise electrically conducting elements for generating electrical potentials at predefined times thereby resulting in an embedded polarity for contributing to the generation of said force for a contraction or an expansion of said sheet 40 to support said ventricular activities of heart 10 in order to prevent failure thereof. In general, sheet 40 possesses a diastolic function and a systolic function in support of said ventricular activities of heart 10. Such a diastolic function can be augmented by the active relaxation of sheet 40. The systolic function can be augmented by a myocarderial contraction of sheet 40.

A controller 60 is generally in communication with said plurality of MEMS elements 44 of said sheet 40 in order to control the contraction of expansion of sheet 40, while a microprocessor 90 is generally in communication with controller 60. Microprocessor 90 and controller 60 can be implemented in the context of a pacemaker 90, which is generally in communication with sheet 40. Microprocessor 90 can be implemented as a central processing unit (CPU) on a single integrated circuit (IC) computer chip. Microprocessor 90 generally functions as the central processing unit of apparatus 70, and can interpret and execute instructions, and generally possesses the ability to fetch, decode, and execute instructions and to transfer information to and from other resources over a data-transfer path or bus.

Note that each MEMS element among said plurality of MEMS elements 44 of sheet 40 can contract toward one another in systole and away from one another by a reversal of poles in diastole. Additionally, each MEMS element among said plurality of MEMS elements 44 sequentially contracts heart 10 horizontally and thereafter, vertically. As indicated previously, each MEMS element is electrical insulated. Sheet 40 can be configured from a flexible or pliable material. Sheet 40 can also be implemented as a mesh arrangement of MEMS elements 44.

The embodiments and examples set forth herein are presented to best explain the present invention and its practical application and to thereby enable those skilled in the art to make and utilize the invention. Those skilled in the art, however, will recognize that the foregoing description and examples have been presented for the purpose of illustration and example only. Other variations and modifications of the present invention will be apparent to those of skill in the art, and it is the intent of the appended claims that such variations and modifications be covered.

The description as set forth is not intended to be exhaustive or to limit the scope of the invention. Many modifications and variations are possible in light of the above teaching without departing from the scope of the following claims. It is contemplated that the use of the present invention can involve components having different characteristics. It is intended that the scope of the present invention be defined by the claims appended hereto, giving full cognizance to equivalents in all respects.

The embodiments of the invention in which an exclusive property or right is claimed are defined as follows. Having thus described the invention what is claimed is:

1. A ventricular assist apparatus, comprising:
a sheet of MEMS-based material wrapped around a failing heart, wherein said sheet of MEM-based material contracts or expands in order to support said failing heart and ventricular activities thereof.

2. The apparatus of claim 1 further comprising:
a controller in communication with said sheet of MEMS-based material for controlling a contraction or an expansion of said sheet when said sheet is wrapped around said failing heart; and
a microprocessor in communication with said controller for processing data communicated to and from said controller.

3. The apparatus of claim 2 further comprising a pacemaker that includes said controller and said microprocessor, wherein said pacemaker is in communication with said sheet.

4. A ventricular assist apparatus, comprising:
a sheet comprising a plurality of (MEMS) elements linked to one another; and
wherein each MEMS element of said plurality of MEMS elements comprises an embedded electrical polarity, which contributes to the generation of a force for contraction or expansion by said sheet in order to support ventricular activities of a heart and prevent failure thereof when said sheet is wrapped around said heart.

5. The apparatus of claim 4 wherein said plurality of MEMS elements comprises electrical conducting elements for generating electrical potentials at predefined times thereby resulting in said embedded polarity for contributing to the generation of said force for a contraction or an expansion of said sheet to support said ventricular activities of said heart in order to prevent failure thereof.

6. The apparatus of claim 4 wherein said sheet comprises a diastolic function and a systolic function in support of said ventricular activities of said heart.

7. The apparatus of claim 6 wherein said diastolic function is augmented by an active relaxation of said sheet.

8. The apparatus of claim 6 wherein said systolic function is augmented by a myocarderial contraction of said sheet.

9. The apparatus of claim 4 further comprising:
a controller in communication with said plurality of MEMS elements of said sheet for controlling contraction of expansion of said sheet; and
a microprocessor in communication with said controller.

10. The apparatus of claim 9 further comprising a pacemaker that includes said controller and said microprocessor, wherein said pacemaker is in communication with said sheet.

11. The apparatus of claim 4 wherein each MEMS element among said plurality of MEMS elements of said sheet contract toward one another in systole and away from one another by a reversal of poles thereof in diastole.

12. The apparatus of claim 4 wherein each MEMS element among said plurality of MEMS elements sequentially contracts said heart horizontally and thereafter, vertically.

13. The apparatus of claim 4 wherein each MEMS element among said plurality of MEMS elements is electrical insulated.

14. The apparatus of claim 4 wherein said sheet comprises a flexible material.

15. The apparatus of claim 4 wherein said sheet comprises a pliable material.

16. The apparatus of claim 4 wherein said sheet comprises a sheath.

17. The apparatus of claim 4 wherein said sheet comprises a mesh arrangement of said plurality of MEMS elements.

18. A ventricular assist system, comprising:
a sheet comprising a flexible material composed of a plurality of (MEMS) elements linked to one another, wherein each MEMS element of said plurality of MEMS elements comprises an embedded electrical polarity, which contributes to the generation of a force for contraction or expansion by said sheet in order to support ventricular activities of a heart and prevent failure thereof when said sheet is wrapped around said heart;
said plurality of MEMS elements comprising electrical conducting elements for generating electrical potentials at predefined times thereby resulting in said embedded polarity for contributing to the generation of said force for contraction or expansion by said sheet in order to support said ventricular activities of said heart to prevent failure thereof;
a controller in communication with said plurality of MEMS elements of said sheet for controlling contraction of expansion of said sheet; and
a microprocessor in communication with said controller, wherein said sheet comprises a diastolic function and a systolic function in support of said ventricular activities of said heart, such that said diastolic function is augmented by an active relaxation of said sheet and said systolic function is augmented by a myocarderial contraction of said sheet.

19. The apparatus of claim 18 wherein:
each MEMS element among said plurality of MEMS elements of said sheet contract toward one another in systole and away from one another by a reversal of poles thereof in diastole;
each MEMS element among said plurality of MEMS elements sequentially contracts said heart horizontally and thereafter, vertically.

20. The apparatus of claim 19 wherein each MEMS element among said plurality of MEMS elements is electrical insulated.
